(19) United States
(12) Patent Application Publication
(10) Pub. No.: US 2026/0114897 A1
(43) Pub. Date: Apr. 30, 2026

(54) ADAPTABLE DILATOR AND METHODS OF USING THE SAME

(71) Applicant: Boston Scientific Medical Device Limited, Galway (IE)

(72) Inventors: Charlene Leung, Scarborough (CA); Linus Hoi Che Leung, Toronto (CA); Matthew L. Choi, Markham (CA)

(21) Appl. No.: 19/003,572

(22) Filed: Dec. 27, 2024

Related U.S. Application Data
(60) Provisional application No. 63/616,426, filed on Dec. 29, 2023.

(51) Int. Cl. A61B 17/34; A61B 17/00

(52) U.S. Cl. CPC A61B 17/3417; A61B 17/00234; A61B 17/3403; A61B 2017/00243; A61B 2017/00305; A61B 2017/00477; A61B 2017/00862; A61B 2017/3454

(57) ABSTRACT

An adaptable dilator hub comprises a dilator hub housing defining a hub lumen extending through the dilator hub housing. The dilator hub housing defines an outer diameter, and the hub lumen defines an inner diameter. The dilator hub comprises a locking mechanism disposed within the dilator hub housing, the locking mechanism engages with the hub lumen, and configured to secure a guidewire therein. A hub feature is disposed on an outer surface of the dilator hub housing defining a feature dimeter. The feature diameter being larger than the outer diameter of the hub housing.

### CROSS REFERENCE TO RELATED APPLICATIONS

This application claims priority to U.S. Provisional Patent Application No. 63/616,426 entitled “ADAPTABLE DILATOR AND METHODS OF USING THE SAME,” filed Dec. 29, 2023, which is hereby incorporated by reference in its entirety.

### TECHNICAL FIELD

The present invention relates generally to methods and devices usable to deliver a therapy to a patient. More specifically, the present invention is concerned with a system and method for delivering a therapy device to a heart.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein and directed towards the heart. This device relies on the use of mechanical force to drive the sharp tip through the septum. Alternatively, radiofrequency perforation apparatuses have been developed, whereby the septal perforation is accomplished by the application of focused radiofrequency energy to the septal tissue via an electrode at the distal end of a relatively thin conductive probe.

Such perforation devices are often used in conjunction with a dilator to help support and guide the perforation device. Such dilators are often used in conjunction with a therapy sheath adapted to deliver a therapy to the patient. The perforation means and the therapy means are generally provided utilizing different sheaths, which requires multiple steps to exchange between the two. Reducing the number of exchanges reduces the time of the operation, thereby reducing risks to the patient.

### SUMMARY

In Example 1, a dilator hub for use within a crossing device for facilitating access to a patient's heart for coupling with one or more sheaths, the dilator hub comprising: a dilator hub housing defining a hub lumen extending through the dilator hub housing, wherein the dilator hub housing defines an outer diameter and the hub lumen defines an inner diameter; a locking mechanism, disposed within the dilator hub housing, wherein the locking mechanism engages with the hub lumen such that the locking mechanism is configured to secure a guidewire to the hub lumen; and a hub feature disposed on an outer surface of the dilator hub housing wherein the hub feature defines a feature diameter, wherein the feature diameter is larger than the outer diameter of the dilator hub housing.

Example 2 is the dilator hub of Example 1, wherein the locking mechanism is configured within the hub housing, and wherein the locking mechanism is configured to transition between a locked position and an unlocked position, wherein in the unlocked position the guidewire is movable within the hub lumen, and wherein in the locked position the guidewire is secured within the hub lumen.

Example 3 is the dilator hub of any of Example 1-2, wherein in the unlocked position the hub housing defines a variable outer diameter.

Example 4 is the dilator hub of Examples 2-3, wherein in the locked position the hub housing defines a uniform outer diameter.

Example 5 is the dilator hub of any of Examples 2-4, wherein the locking mechanism positions the guidewire against an inner surface of the hub lumen.

Example 6 is the dilator hub of any of Examples 2-4, wherein the locking mechanism secures the guidewire centrally within the hub lumen.

Example 7 is the dilator hub of any of Examples 1-6, wherein the hub feature is a cap, wherein the cap is configured to engage with the outer surface of the dilator hub housing, to prevent disengagement of a therapy sheath.

Example 8 is the dilator hub of Example 7, wherein the cap defines a protrusion and the dilator hub housing defines a cavity to receive the protrusion.

Example 9 is the dilator hub of Example 7, wherein the dilator hub housing defines a protrusion and the cap defines a cavity to receive the protrusion.

Example 10 is the dilator hub of Example 7, wherein the cap is configured to engage in a frictional engagement with the outer surface of the dilator hub housing.

Example 11 is the dilator hub of Example 7, wherein the cap and the dilator hub housing component define a threaded connection.

Example 12 is the dilator hub of any of Examples 1-6, wherein the hub feature comprises an elastic material such that the hub feature is deformable to receive a therapy sheath.

Example 13 is the dilator hub of any of Examples 1-6, wherein the hub feature element defines an exoskeleton defining a collapsible frame, wherein the exoskeleton comprises nickel titanium.

Example 14 is the dilator hub of any of Examples 1-6, wherein the hub feature comprises an expanding collet defining a variable collet diameter, wherein the variable collet diameter is variable between a contracted diameter and an expanded diameter, wherein the contracted diameter is smaller than a therapy sheath inner diameter, and the expanded diameter is larger than the therapy sheath inner diameter.

Example 15 is the dilator hub of any of Examples 1-14, wherein the guidewire comprises an RF tip.

In Example 16, a dilator hub is used within a crossing device for facilitating access to a patient's heart for coupling with one or more sheaths, the dilator hub comprising: a dilator hub housing defining a hub lumen extending through the dilator hub housing, wherein the dilator hub housing defines an outer diameter and the hub lumen defines an inner diameter; a locking mechanism, disposed within the dilator hub housing, wherein the locking mechanism engages with the hub lumen such that the locking mechanism is configured to secure a guidewire to the hub lumen; and a hub feature disposed on an outer surface of the dilator hub housing wherein the hub feature defines a feature diameter, wherein the feature diameter is larger than the outer diameter of the dilator hub housing.

In Example 17, the dilator hub of Example 16, wherein the locking mechanism is configured within the hub housing, and wherein the locking mechanism is configured to transition between a locked position and an unlocked position, wherein in the unlocked position the guidewire is movable within the hub lumen, and wherein in the locked position the guidewire is secured within the hub lumen.

In Example 18, the dilator hub of Example 17, wherein in the unlocked position the hub housing defines a variable outer diameter.

In Example 19, the dilator hub of Example 17, wherein in the locked position the hub housing defines a uniform outer diameter.

In Example 20, the dilator hub of Example 17, wherein the locking mechanism positions the guidewire against an inner surface of the hub lumen.

In Example 21, the dilator hub of Example 16, wherein the locking mechanism secures the guidewire centrally within the hub lumen.

In Example 22, the dilator hub of Example 16, wherein the hub feature is a cap, wherein the cap is configured to engage with the outer surface of the dilator hub housing, to prevent disengagement of a therapy sheath.

In the Example 23, the dilator hub of Example 22, wherein the cap defines a protrusion and the dilator hub housing defines a cavity to receive the protrusion.

In the Example 24, the dilator hub of Example 22, wherein the dilator hub housing defines a protrusion and the cap defines a cavity to receive the protrusion.

In the Example 25, the dilator hub of Example 22, wherein the cap is configured to engage in a frictional engagement with the outer surface of the dilator hub housing.

In Example 26, the dilator hub of Example 22, wherein the cap and the dilator hub housing component define a threaded connection.

In Example 27, the dilator hub of Example 16, wherein the hub feature comprises an elastic material such that the hub feature is deformable to receive a therapy sheath.

In Example 28, the dilator hub of Example 16, wherein the hub feature element defines an exoskeleton defining a collapsible frame, wherein the exoskeleton comprises nickel titanium.

In Example 29, the dilator hub of Example 16, wherein the hub feature comprises an expanding collet defining a variable collet diameter, wherein the variable collet diameter is variable between a contracted diameter and an expanded diameter, wherein the contracted diameter is smaller than a therapy sheath inner diameter, and the expanded diameter is larger than the therapy sheath inner diameter.

In Example 30, the dilator hub of Example 16, wherein the hub feature is at least one ramp disposed on the outer surface the dilator hub housing, wherein the at least one ramp defines a proximal side and a distal side, and wherein the at least one ramp increases in diameter from the proximal side to the distal side.

In Example 31, an assembly for facilitating access to a patient's heart comprises: a dilator hub, the dilator hub comprising: a hub housing defining a hub lumen extending through the hub, wherein the dilator hub housing defines an outer diameter and the hub lumen defines an inner diameter; a locking mechanism, disposed within the hub housing wherein the locking mechanism is configured to secure a guidewire to the hub lumen; a hub backloading element disposed about the hub housing wherein the hub backloading element defines an element diameter, wherein the element diameter is different than the outer diameter of the hub and the inner diameter of the hub; a dilator shaft, defining a dilator lumen extending between a distal end and a proximal end, wherein the proximal end engages with the dilator hub, wherein the distal end comprises a transition section defining a variable outer diameter, and wherein the dilator lumen is adapted to receive and support a puncturing device; and a therapy sheath configured to extend over the dilator hub and the dilator lumen.

In Example 32, the assembly of Example 31, wherein the hub backloading element comprises a loading configuration and a maintenance configuration, wherein in the loading configuration the therapy sheath is moveable over the dilator hub, and wherein in the maintenance configuration the therapy sheath is not removable over the dilator hub.

In Example 33, a method of providing therapy access within a patient's body, the method comprising: accessing a region of tissue within the patient's body using an assembly, the assembly comprising a dilator hub, the dilator hub comprising: a dilator hub housing defining a hub lumen extending through the hub, wherein the dilator hub housing defines an outer diameter and the hub lumen defines an inner diameter; a locking mechanism, disposed within the dilator hub housing wherein the locking mechanism is configured to secure a guidewire to the hub lumen; a hub backloading element disposed about the hub housing wherein the hub backloading element defines an element diameter, wherein the element diameter is different than the outer diameter of the hub and the inner diameter of the hub; a dilator shaft, defining a dilator lumen extending between a distal end and a proximal end, wherein the proximal end engages with the dilator hub, wherein the distal end comprises a transition section defining a variable outer diameter, and wherein the dilator lumen is adapted to receive and support a puncturing device; and a therapy sheath configured to extend over the dilator hub and the dilator lumen; positioning the guidewire through the dilator hub into the dilator lumen forming an entry device; advancing the entry device to towards the target tissue; advancing a puncture device from the guidewire; puncturing the target tissue at a target site with the puncture device; locking the guidewire within the hub lumen; loading the therapy sheath over the dilator hub and dilator shaft; and advancing the therapy sheath to the target tissue site.

In Example 34, the method of Example 33, further comprising changing the hub backloading element from a loading position to a maintenance position thereby preventing movement of the therapy sheath over the dilator hub.

In an Example 35, the method of Example 34, wherein the hub backloading element defines a loading outer diameter and a maintenance outer diameter, wherein the loading outer diameter is smaller than an inner diameter of the therapy sheath, and the maintenance outer diameter is larger than the inner diameter of the therapy sheath.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the invention.

FIGS. 2A-2B are perspective views of a dilator and sheath according to embodiments of the invention.

FIG. 3 is a schematic view of the transseptal access system according to embodiments of the invention.

FIG. 4A is a perspective view of an example dilator hub housing including a locking device in an unlocked position according to embodiments of the invention.

FIG. 4B is a sectional view of the example locking device in an unlocked position and FIG. 4C is a sectional view of the locking device in the locked position according to embodiments of the present invention.

FIG. 5A is a sectional view of another example dilator hub locking device in an unlocked position and FIG. 5B is a sectional view of the example locking device in a locked position according to embodiments of the present invention.

FIG. 6A is a perspective view of an example hub housing feature prior to engagement with a therapy sheath and FIG. 6B is a perspective view of the example hub housing feature engaged with the therapy sheath according to embodiments of the present invention.

FIG. 7A is a sectional view of another example hub housing feature in a disengaged configuration and FIG. 7B is a sectional view of the hub housing feature in an engaged configuration according to embodiments of the present invention.

FIG. 8 is a sectional view of another example hub housing feature in a disengaged configuration according to embodiments of the present invention.

FIG. 9 is a sectional view of another example hub housing feature in a disengaged configuration according to embodiments of the present invention.

FIG. 10 is a perspective view of another example hub housing feature according to embodiment of the present invention.

FIG. 11A is a sectional view of another example hub housing feature in a disengaged configuration and FIG. 11B is a sectional view of the hub housing feature element in an engaged configuration according to embodiments of the present invention.

While the invention is amenable to various modifications and alternative forms, specific embodiments have been shown by way of example in the drawings and are described in detail below. The intention, however, is not to limit the invention to the embodiments described. On the contrary, the invention is intended to cover all modifications, equivalents, and alternatives falling within the scope of the invention as defined by the appended claims.

### DETAILED DESCRIPTION

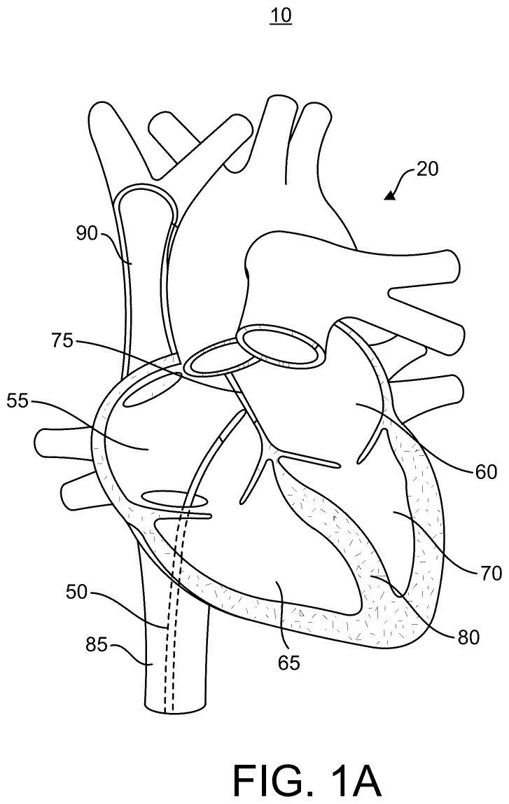

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes a dilator sheath 100, a dilator 105 having a dilator body 107 and a tapered distal tip portion 108, and a perforation device (e.g., a radiofrequency (RF) perforation device) 110 having distal end portion 112 terminating in a tip electrode 115. As shown, in the assembled use state illustrated in FIGS. 1A-1C, the RF perforation device 110 can be disposed within the dilator 105, which itself can be disposed within the dilator sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The dilator sheath 100 may then be introduced into the femoral vein over the guidewire, and advanced towards the heart 20. In one embodiment, the distal ends of the guidewire and dilator sheath 100 are then positioned in the SVC 90. These steps may be performed with the aid of an imaging system, e.g., fluoroscopy or ultrasonic imaging. The dilator 105 may then be introduced into the dilator sheath 100 and over the guidewire, and advanced through the dilator sheath 100 into the SVC 90. Alternatively, the dilator 105 may be fully inserted into the dilator sheath 100 prior to entering the body, and both may be advanced simultaneously towards the heart 20. When the guidewire, dilator sheath 100, and dilator 105 have been positioned in the SVC 90, the guidewire is removed from the body, and the dilator sheath 100 and the dilator 105 are retracted so that their distal ends are positioned in the right atrium 55. The RF perforation device 110 described can then be introduced into the dilator 105, and advanced toward the heart 20. In some embodiments, the guidewire may include the RF perforation device 10.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Boston Scientific Medical Device, LTD. The aforementioned pre-formed shapes can advantageously function to minimize the risk of unintended contact between the tip electrode 115 and tissue within the left atrium 60, and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

In certain embodiments, catheters, therapy devices and sheaths can be deployed through the sheath 100, after it is successfully deployed into the desired heart chamber (e.g., the left atrium). In other embodiments, the therapy device (e.g., mapping catheter, therapy sheath, medical device, etc.) is part of the sheath 100, creating a therapy sheath.

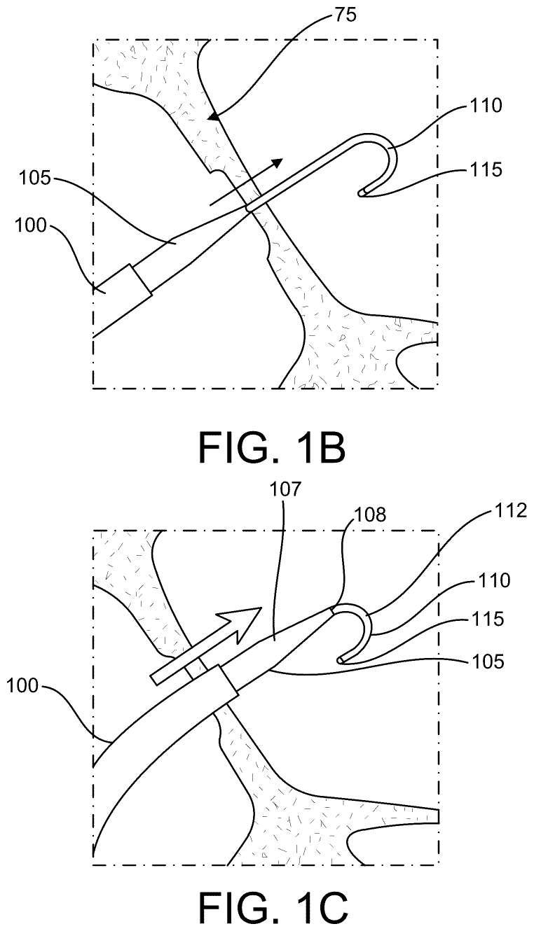

FIGS. 2A-2B are perspective views of a dilator and sheath according to known embodiments of a dilator inserted into a sheath. As shown, the dilator 105 is partially inserted into a lumen of the sheath 100. The dilator 105 includes a handle 201, a dilator hub 202 and a dilator shaft 204. The sheath 100 includes a sheath hub 206 and a sheath body 208. As illustrated, the dilator 105, at least, the handle 201 thereof must be removed prior to inserting or removing additional sheaths, for example, a therapy sheath.

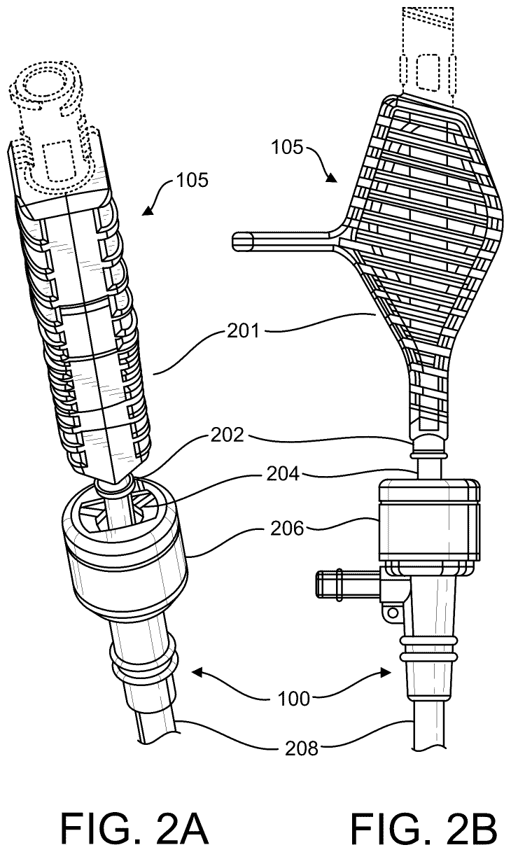

FIG. 3 illustrates an assembly 300 for facilitating access to a patient's heart. The assembly includes an adaptable dilator hub 320, configured to interact with a dilator sheath 305 and a therapy sheath 355. A guidewire 318 may extend through the dilator sheath 305 from a proximal side of the dilator lumen to a distal side. In some embodiments, the guidewire 318 may comprise a perforation device 310 on the distal end configured to perforate the fossa ova to provide access to the desired therapy site. In some embodiments, the guidewire 318 may extend from a tapered distal tip of the dilator sheath 305, in this regard, the distal tip of the dilator sheath 305 may define a smaller diameter than the rest of the dilator sheath 305. In some embodiments, the transition area 330 between the dilator tip 308 and the guidewire 318 may comprise a zero-transition tip, to aid in positioning the therapy sheath 355 at the target location. In this regard, the zero-transition tip may provide a seamless transition between the guidewire and the dilator tip 308 to provide a smooth transition point for entry and removal of the assembly 300.

As illustrated in FIG. 3 the therapy sheath 355 may be configured to be positioned directly over the adaptable dilator hub 320 without removing the dilator sheath 305, the adaptable dilator hub 320, and/or the guidewire 318. As will be discussed herein the adaptable dilator hub 320, and the transition area 330 comprise features to secure the components of the assembly together, and to maintain the position of the assembly within a patient allow application of therapy to the target site in fewer steps.

In order to facilitate the application of therapy in a reduced number of steps, the adaptable dilator comprises a locking mechanism, and a hub feature to provide backloading of the therapy sheath, both features allow the therapy sheath to be positioned about the dilator without removal of the guidewire or the dilator prior to insertion of the therapy sheath.

In order to successfully apply a therapy sheath over the dilator the assembly may be stably positioned within the patient, and the components of the assembly must be anchored. Guidewires may be utilized navigate the device to the target location and may facilitate the puncture of the fossa ovalis to allow the therapy sheath to be positioned in the correct position to provide the desired therapy to the target area. As such, in some embodiments, the guidewire may include a perforation device at the tip (e.g., a RF tip) and is thus, generally removed prior to insertion of the therapy sheath, to prevent accidental perforations. In this regard, if the guidewire is not removed prior to insertion of the therapy sheath, it may be desirable to secure the guidewire within the assembly to prevent movement which may cause accidental perforations.

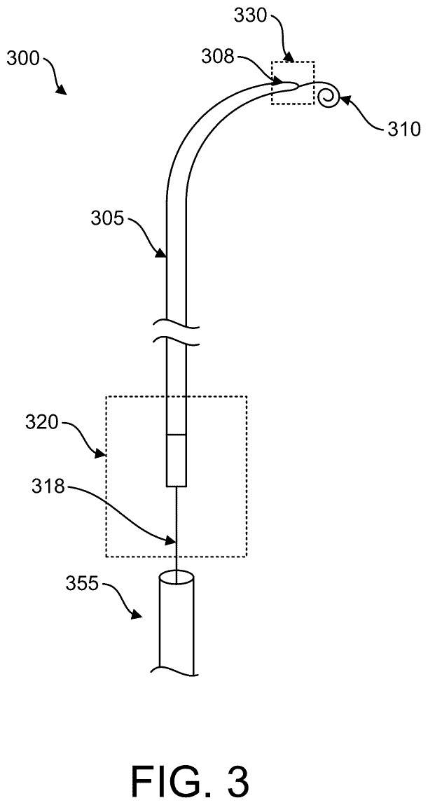

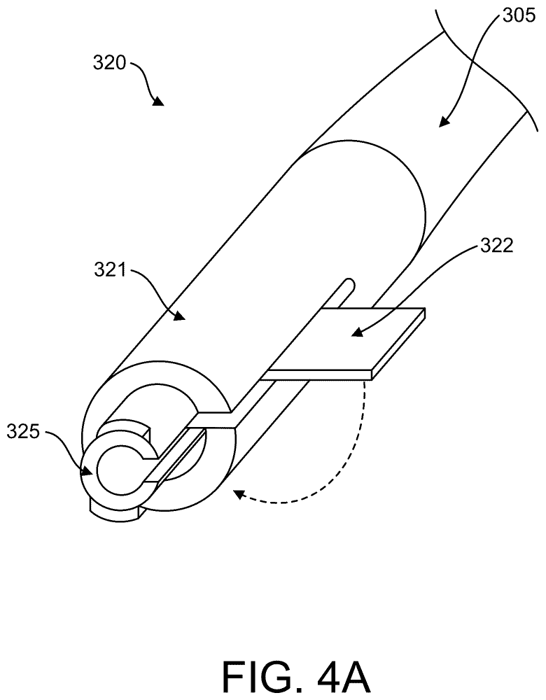

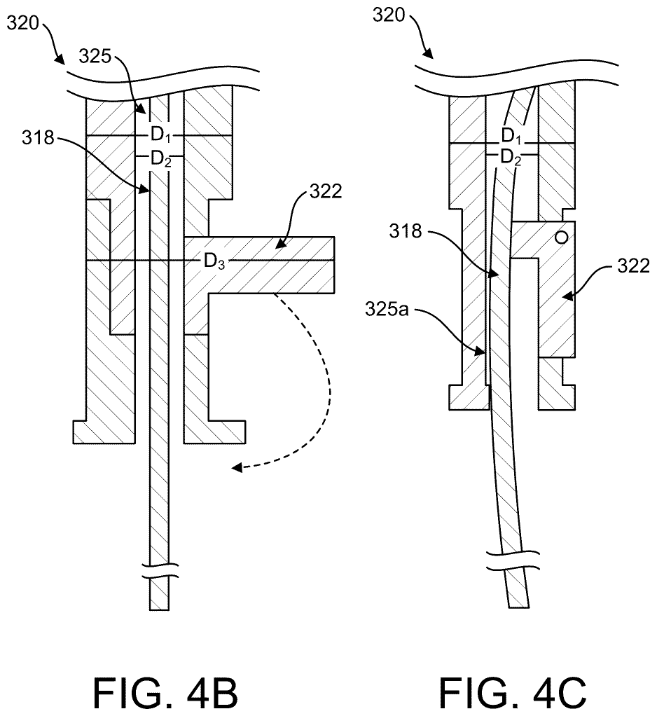

FIGS. 4A-5B illustrate locking mechanisms to secure a guidewire to the assembly. FIGS. 4A-4C illustrate an example locking mechanism 322 of a dilator hub 320. In some embodiments, the dilator hub 320 comprises a dilator hub housing 321 defining a hub lumen 325 extending there through. The dilator hub housing 321, as illustrated in FIG. 4B may define an outer diameter D₁, and the hub lumen 325 may define an inner diameter D₂.

In some embodiments, the locking mechanism 322 may be transitional between an unlocked state, illustrated in FIGS. 4A-B, and a locked state, illustrated in FIG. 4C. In the unlocked state the dilator hub housing 321 may define a variable outer diameter. In this regard the dilator hub housing 321 may not define a uniform diameter throughout the housing, for example, the housing outer diameter D₁ may be smaller than a locking outer dimeter D₃, defined when the locking mechanism 322 is in the unlocked position.

In the illustrated embodiment, the locking mechanism 322 may be configured as a lever formed within the dilator hub housing 321. The lever may transition between an unlocked position, wherein the lever does not interact with the guidewire 318, and thus, the guidewire may move freely within the hub lumen 325 (e.g., about the circumference of the lumen, and longitudinally within the lumen), and a locked position, where the guidewire 318 is secured to and/or within the hub lumen 325. When the lever is transitioned into the locked position the lever may secure the guidewire 318 within the hub lumen 325, specifically, against an inner wall 325a.

In some embodiments, the guidewire 318 may be secured within the hub lumen 325, while in other embodiments, the guidewire 318 may be secured outside of the hub lumen 325. Securing the guidewire 318 within the dilator hub lumen 325 prevents movement of the guidewire 318, and therefore prevents movement of the assembly within a patient. Once the guidewire 318 is secured the system is anchored within the patient, and additional sheaths and/or wires may be advanced to provide the desired therapy to the target area.

In some embodiments, in the locked position the dilator hub housing 321 may define a constant outer diameter D₁. In some embodiments, in the locked position the dilator hub housing 421 may defined a smooth surface, such that a therapy sheath, or other sheath may be positioned over the outer surface of the dilator hub housing, thereby preventing loading disruptions.

FIGS. 5A-B illustrate an alternative locking mechanism, wherein the locking mechanism is configured as a pad 323. In some embodiments, the pad 323 may be one pad which extends about the circumference of the hub lumen 325, while in other embodiments, the pad 323 may be configured as more than one pad positioned within the hub lumen 325, wherein the more than one pad is configured to extend about the circumference of the hub lumen 325. In some embodiments, the pad 323 may be configured to secure the guidewire 318 within the hub lumen 325 against the pad 323, rather than against the inner surface of the hub lumen 325. In some embodiments, the pad 323 may be transitional along a lumen axis. In this regard, the pad 323 may be formed independent of the dilator hub housing 321. In some embodiments, the pad 323 may be removable from the dilator hub housing 321.

In this regard, the pad 323 may be circumferentially positioned about the guidewire 318 and may be moved to form an interference fit with the hub lumen 325. In some embodiments, the pad 323 may comprise one or more steps on an outer surface 323a wherein the one or more steps may engage with the hub housing 321. In some embodiments, in the unlocked position, illustrated in FIG. 5A, the pads 323 may be positioned such that an inner pad diameter D₄ is equivalent to the inner diameter D₂ of the hub lumen 325, while an outer pad diameter D₅ is equivalent or greater than the outer diameter D₁ of the dilator hub housing 321. In the locked position, illustrated in FIG. 5B, the pad(s) 323 may be moved at least partially within the hub lumen 325 such that an inner surface 323b of the pads 323 surrounds and is in contact with the guidewire 318, thereby anchoring the guidewire 318 in place.

In some embodiments, a key 324 may be used to move the pads 323 into place about the guidewire 318. In some embodiments, the key 324 may define a diameter less than the outer diameter D₁ of the dilator hub housing 322, while in other embodiments, the key 324 may be removable and/or not continuous about the guidewire 318.

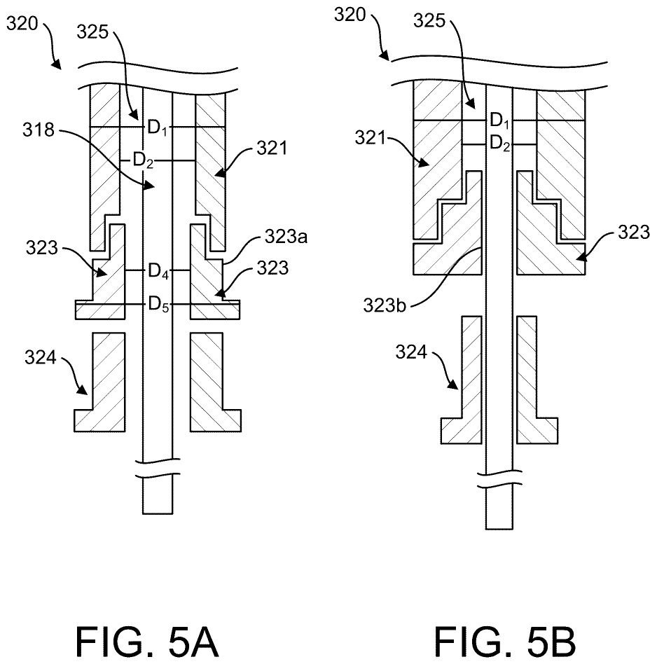

After securing the guidewire to the dilator hub housing, a therapy sheath may be backloaded over the dilator hub housing to position the therapy sheath at the target site. In this regard, the dilator hub housing may be configured to facilitate backloading of the therapy sheath without removing the entire dilator hub, specifically as the dilator hub may be being utilized to secure the guidewire and thereby anchor the assembly. In this regard, the dilator hub housing may comprise a hub feature disposed on an outer surface of the dilator hub housing which allows backloading, and in some embodiments, prevents rotation and/or undesired movements of the therapy sheath after backloading. FIGS. 6A-10B illustrate example hub features.

FIGS. 6A-B illustrate an example hub feature configured as a ramp 431. In some embodiments, the ramp 431 may positioned on the proximal side of a dilator hub housing 421. In some embodiments, the ramp 431 may be positioned adjacent the proximal side of the dilator hub housing 421, while in other embodiments the ramp 431 may be attached to and extend from the dilator hub housing 421. In some embodiments, the ramp 431 may be positioned continuous about the circumference of the distal hub housing 421, while in other embodiments, the ramp 431 may be discontinuous over the entire circumference of the distal hub housing 421, for example, the ramp 431 may define two distinct ramp portions diametrically opposed to one another about the dilator hub housing 421.

In some embodiments, the ramp 431 may define a variable diameter, wherein the diameter increases between the proximal side ramp, to a distal side of the ramp, wherein the distal side of the ramp is adjacent to the dilator hub housing 421. In this regard, the distal side of the ramp 431 may define an element D₇, which in some embodiments may be larger than the outer diameter (e.g., D₁ of FIG. 4A) of the dilator hub housing 421.

In some embodiments, the ramp 431 may be an elastic material configured to compress to receive a distal end 455a of a therapy sheath 455. The compressibility of the ramp 431 may provide multiple benefits to the assembly 400. For example, the compressibility, may allow the distal end 455a of the therapy sheath 455 to be positioned over the ramp 431 when the distal end 455a comprises a smaller opening than a proximal end 455a. Thus, the smaller diameter of the ramp 431 may provide a gentle slope for the proximal end 455a to advance over and to compress the ramp 431.

Additionally or alternatively, the ramp 431 may provide stability for the therapy sheath 455 after advancement. In this regard, the ramp 431 may provide an interference fit, or frictional fit between the therapy sheath 455 and the dilator hub housing 421 which may prevent accidental lateral (e.g., along the dilator lumen) or rotational movement about the dilator lumen 405 and dilator hub housing 421, thereby preventing undesired movement.

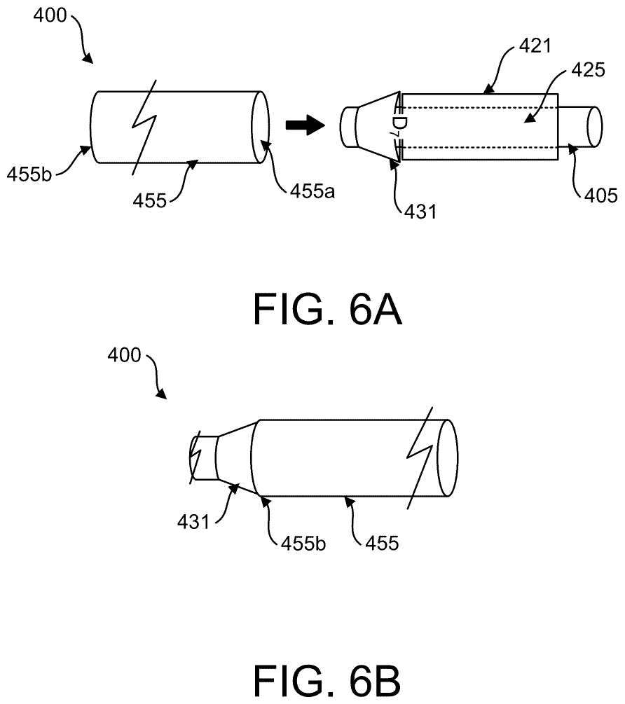

FIGS. 7A-7B illustrate another hub feature to provide backloading of a therapy sheath over the dilator hub housing. In some embodiments, a cap 427 may be removably engageable with the proximal end of the dilator hub housing 421. The dilator hub housing 421 may, as discussed above, define an outer diameter (see e.g., D₁ FIG. 4A), such that a therapy sheath, may be positioned over the dilator hub housing 421 and advanced to the therapy site. To secure the therapy sheath at the target location the cap 427 may be locked to the dilator hub housing 421 to prevent axial movement, and rotational movement of the therapy sheath. In some embodiments, to prevent lateral movement of the therapy sheath over the dilator hub housing 421, the cap 427 may define a feature diameter D₇ which is greater than the inner diameter of the therapy sheath, in this regard, the size of the cap 427 may prevent slippage and/or removal of the therapy sheath prior to the desired time.

In some embodiments, the dilator hub housing 421 may define at least one cavity 432a, and the cap 427 may define at least one protrusion 432b corresponding to the at least one cavity 432a. In this regard, when the cap 427 is positioned onto the dilator hub housing 421 the at least one cavity 432a and the at least one protrusion 432b may lock together to secure the cap 427 to the dilator hub housing 421. In some embodiments, the at least one cavity 432a may extend about the circumference of the dilator hub housing 421, while in other embodiments, the at least one cavity 432a may be formed as one or more discrete cavities about the circumference of the dilator hub housing 421. In some embodiments, the at least one cavity 432a may be positioned in a linear arrangement about the circumference of the dilator hub housing 421, while in other embodiments the at least one cavity 432a may not be linear about the circumference of the dilator hub housing 421.

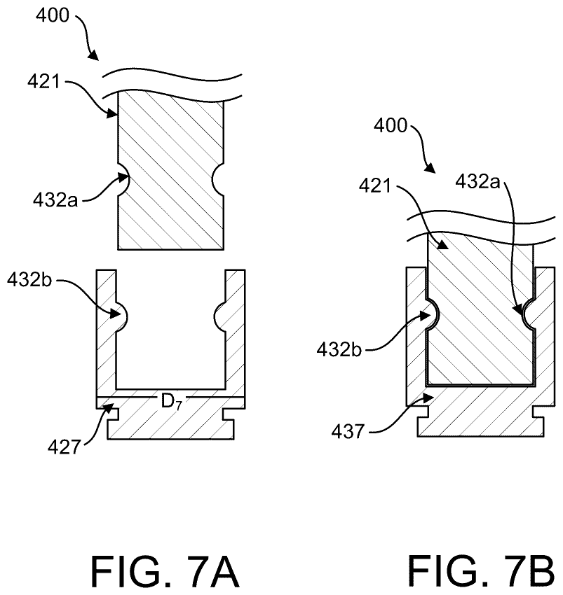

In an alternative embodiment, illustrated in FIG. 8, the dilator hub housing 421 may define at least one hub protrusion 433a, and the cap 427 may define at least one cap cavity 433b. The at least one hub protrusion 433a and the at least one cap cavity 433b may lock together to secure the cap 427 to the dilator hub housing 421.

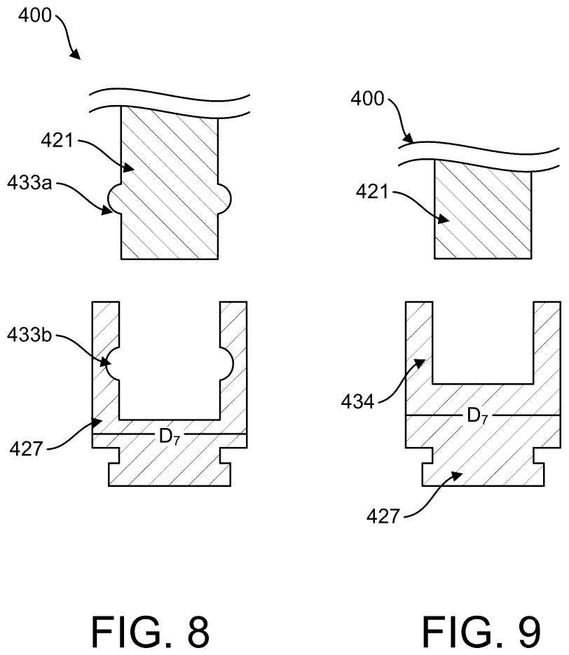

In yet another embodiment, illustrated in FIG. 9, the dilator hub housing 421 and the cap 427 may each define smooth surfaces. In the regard, the cap 427 and the dilator hub housing 421 may form a friction fit to secure the cap 427 onto the dilator hub housing 421. Although, a friction fit, and bump and lock fit are illustrated, other connection mechanisms, for example, twistable, snappable, and other similar fits are contemplated.

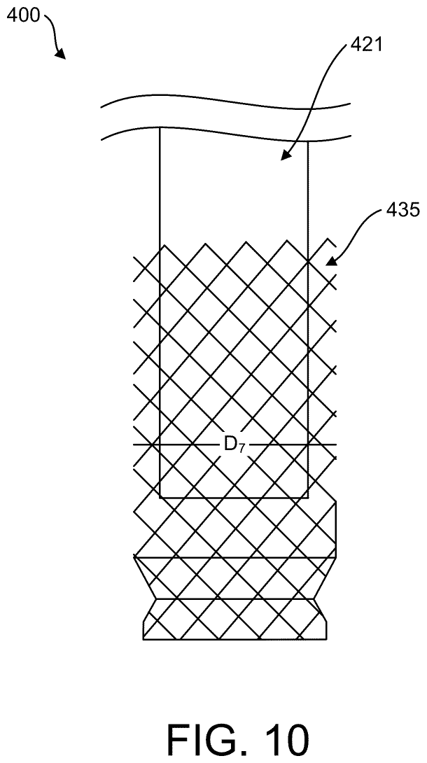

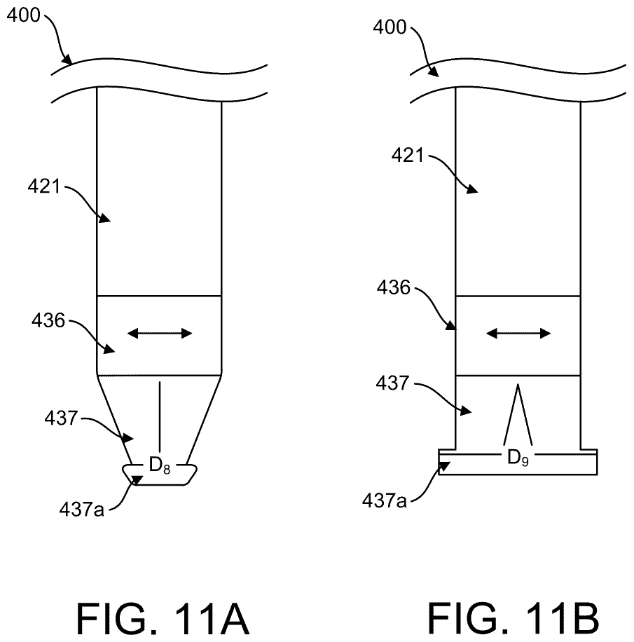

In yet another example embodiment, illustrated in FIGS. 10-11B, the dilator hub housing 421 may utilize a collapsing mechanism to allow the therapy sheath to be loaded over the dilator hub housing 421. FIG. 10 illustrates an example embodiment where the dilator hub housing 421 is surrounded by a collapsing mechanism 435. In some embodiments, the collapsing mechanism 435 may include a collapsible frame, or exoskeleton. In this regard, the collapsing mechanism may deformable. The collapsing mechanism 435 may be configured to compress and expand between a compressed state and an expanded state. In the compressed state the collapsing mechanism 435 may define a smaller diameter, such as to receive the tip of the therapy sheath. Upon insertion into the therapy sheath, and the expansion of the inner diameter of the therapy sheath from the therapy sheath tip to the therapy sheath lumen, the collapsing mechanism 435 may expand to fill the larger space of the lumen of the therapy sheath, while preventing rotation about the dilator hub housing. In some embodiments, the collapsing mechanism 435 may be formed of a nickel titanium frame with a fabric filler or wall inner structure.

In some embodiments, the collapsing mechanism 435 may comprise a feature diameter D₇ measured when the collapsing mechanism 435 is at a neutral state. In this regard, the feature diameter D₇ may be larger than the outer diameter (e.g., D₁ of FIG. 4A) and smaller than a therapy sheath diameter. The feature diameter D₇ may compress to a smaller diameter to allow insertion of the therapy sheath over the dilator hub.

In another embodiment, illustrated in FIGS. 11A-B, the collapsing mechanism may be formed as a chuck and collet system. In this regard the system may be disposed on the proximal side of the dilator hub housing 421. The system 400 may comprise a rotating feature 436 in mechanical connection with a collet 437. The collet 437 may define a collet tip 437a which defines a varying diameter. In this regard, rotation of the rotating feature 436 causes the collet tip 437a to expand or contract depending on the direction of rotation. For example, rotating of the rotating feature a clockwise direction may cause the collet tip 437a to expand, while rotating the collet tip 437b counterclockwise may cause the collet tip 437b to contract. The collet tip 437a may extend between a contracted diameter D₈, wherein the contracted diameter D₈ is smaller than the therapy sheath tip diameter to an expanded diameter D₉, wherein the expanded diameter Do is equal to or larger than the therapy sheath diameter. Thus, in the contracted position the therapy sheath tip may be positioned over the chuck and collet system, and while expanded the therapy sheath may be retained on the dilator hub housing 421.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A dilator hub for use within a crossing device for facilitating access to a patient's heart for coupling with one or more sheaths, the dilator hub comprising:
a dilator hub housing defining a hub lumen extending through the dilator hub housing, wherein the dilator hub housing defines an outer diameter and the hub lumen defines an inner diameter;
a locking mechanism, disposed within the dilator hub housing, wherein the locking mechanism engages with the hub lumen such that the locking mechanism is configured to secure a guidewire to the hub lumen; and
a hub feature disposed on an outer surface of the dilator hub housing wherein the hub feature defines a feature diameter, wherein the feature diameter is larger than the outer diameter of the dilator hub housing.

2. The dilator hub of claim 1, wherein the locking mechanism is configured within the hub housing, and wherein the locking mechanism is configured to transition between a locked position and an unlocked position, wherein in the unlocked position the guidewire is movable within the hub lumen, and wherein in the locked position the guidewire is secured within the hub lumen.

3. The dilator hub of claim 2, wherein in the unlocked position the hub housing defines a variable outer diameter.

4. The dilator hub of claim 2, wherein in the locked position the hub housing defines a uniform outer diameter.

5. The dilator hub of claim 2, wherein the locking mechanism positions the guidewire against an inner surface of the hub lumen.

6. The dilator hub of claim 1, wherein the locking mechanism secures the guidewire centrally within the hub lumen.

7. The dilator hub of claim 1, wherein the hub feature is a cap, wherein the cap is configured to engage with the outer surface of the dilator hub housing, to prevent disengagement of a therapy sheath.

8. The dilator hub of claim 7, wherein the cap defines a protrusion and the dilator hub housing defines a cavity to receive the protrusion.

9. The dilator hub of claim 7, wherein the dilator hub housing defines a protrusion and the cap defines a cavity to receive the protrusion.

10. The dilator hub of claim 7, wherein the cap is configured to engage in a frictional engagement with the outer surface of the dilator hub housing.

11. The dilator hub of claim 7, wherein the cap and the dilator hub housing component define a threaded connection.

12. The dilator hub of claim 1, wherein the hub feature comprises an elastic material such that the hub feature is deformable to receive a therapy sheath.

13. The dilator hub of claim 1, wherein the hub feature element defines an exoskeleton defining a collapsible frame, wherein the exoskeleton comprises nickel titanium.

14. The dilator hub of claim 1, wherein the hub feature comprises an expanding collet defining a variable collet diameter, wherein the variable collet diameter is variable between a contracted diameter and an expanded diameter, wherein the contracted diameter is smaller than a therapy sheath inner diameter, and the expanded diameter is larger than the therapy sheath inner diameter.

15. The dilator hub of claim 1, wherein the hub feature is at least one ramp disposed on the outer surface the dilator hub housing, wherein the at least one ramp defines a proximal side and a distal side, and wherein the at least one ramp increases in diameter from the proximal side to the distal side.

16. An assembly for facilitating access to a patient's heart, the assembly comprising:
a dilator hub, the dilator hub comprising:
a hub housing defining a hub lumen extending through the hub, wherein the dilator hub housing defines an outer diameter and the hub lumen defines an inner diameter;
a locking mechanism, disposed within the hub housing wherein the locking mechanism is configured to secure a guidewire to the hub lumen;
a hub backloading element disposed about the hub housing wherein the hub backloading element defines an element diameter, wherein the element diameter is different than the outer diameter of the hub and the inner diameter of the hub;
a dilator shaft, defining a dilator lumen extending between a distal end and a proximal end, wherein the proximal end engages with the dilator hub, wherein the distal end comprises a transition section defining a variable outer diameter, and wherein the dilator lumen is adapted to receive and support a puncturing device; and
a therapy sheath configured to extend over the dilator hub and the dilator lumen.

17. The assembly of claim 16, wherein the hub backloading element comprises a loading configuration and a maintenance configuration, wherein in the loading configuration the therapy sheath is moveable over the dilator hub, and wherein in the maintenance configuration the therapy sheath is not removable over the dilator hub.

18. A method of providing therapy access within a patient's body, the method comprising:
accessing a region of tissue within the patient's body using an assembly, the assembly comprising a dilator hub, the dilator hub comprising:
a dilator hub housing defining a hub lumen extending through the hub, wherein the dilator hub housing defines an outer diameter and the hub lumen defines an inner diameter;
a locking mechanism, disposed within the dilator hub housing wherein the locking mechanism is configured to secure a guidewire to the hub lumen;
a hub backloading element disposed about the hub housing wherein the hub backloading element defines an element diameter, wherein the element diameter is different than the outer diameter of the hub and the inner diameter of the hub;
a dilator shaft, defining a dilator lumen extending between a distal end and a proximal end, wherein the proximal end engages with the dilator hub, wherein the distal end comprises a transition section defining a variable outer diameter, and wherein the dilator lumen is adapted to receive and support a puncturing device; and
a therapy sheath configured to extend over the dilator hub and the dilator lumen;
positioning the guidewire through the dilator hub into the dilator lumen forming an entry device;
advancing the entry device to towards the target tissue;
advancing a puncture device from the guidewire;
puncturing the target tissue at a target site with the puncture device;
locking the guidewire within the hub lumen;
loading the therapy sheath over the dilator hub and dilator shaft; and
advancing the therapy sheath to the target tissue site.

19. The method of claim 18, further comprising changing the hub backloading element from a loading position to a maintenance position thereby preventing movement of the therapy sheath over the dilator hub.

20. The method of claim 19, wherein the hub backloading element defines a loading outer diameter and a maintenance outer diameter, wherein the loading outer diameter is smaller than an inner diameter of the therapy sheath, and the maintenance outer diameter is larger than the inner diameter of the therapy sheath.
